(19) United States
(12) Patent Application Publication
(10) Pub. No.: US 2006/0127489 A1
(43) Pub. Date: Jun. 15, 2006

(54) Targeted delivery

(76) Inventors: Michael Crothers, London (GB); Gordon Nelson, Cheshire (GB)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 10/532,368

(22) PCT Filed: Oct. 23, 2003
(86) PCT No.: PCT/GB03/04554

§ 371 Date: Sep. 12, 2005

(30) Foreign Application Priority Data
Oct. 24, 2002 (GB) 0224718.7
Sep. 6, 2003 (GB) 0320952.5

(51) Int. Cl. A61K 9/50; A61K 9/16; C12P 21/06

(52) U.S. Cl. 424/490; 435/69.1

(57) ABSTRACT

An encapsulated product comprises a plurality of micro-capsules formed from a plurality of micro-organisms and having a lipophilic active encapsulated and passively retained within said micro-capsules. The lipophilic active is not being a natural constituent of the micro-organisms, and the micro-capsules has: (a) an at least substantially intact cell wall, and (b) an intact cell membrane. The micro-capsules are formulated to target delivery of the micro-capsules and the lipophilic active to at least one desired mucous membrane.

The present invention relates to microbially encapsulated products, microbial encapsulation, and the targeted delivery and controlled release of actives using microbial microcapsules.

According to the US Food and Drug Administration's (IDA's) Biopharmaceutics Classification System (BCS), drug products are classified into four groups based on the ability of a given drug substance to permeate biological membranes and its aqueous solubility: Class I drugs are highly permeable, highly soluble; Class II drugs are highly permeable, poorly soluble; Class III drugs are poorly permeable, highly soluble; and Class IV drugs are poorly permeable, poorly soluble (The biopharmaceutics classification system (BCS) guidance, Center for Drug Evaluation and Research, US Food and Drug Administration (FDA), 2001, www.fda.gov/cder). A drug substance is considered ‘highly soluble’ when the highest dose strength is soluble in 250 ml water over a pH range 1 to 7.5, and ‘highly permeable’ when the extent of absorption in humans is determined to be 90% of an administered dose, based on mass balance or related to an intravenous reference dose. For a rapidly dissolving tablet, 85% of the labelled amount of drug substance must dissolve within 30 minutes. Thus, for rapidly dissolving solid oral dosage forms, the dose-to solubility ratio (D:S) of the drug must be 250 ml over a pH range of 1 to 7.5. Class I drug substances, which possess both high permeability through biological membranes and good solubility in water, have the preferred physicochemical properties. Most new chemical identities are water-insoluble lipophilic compounds or, in other words, Class II or Class IV compounds which are traditionally difficult to formulate into usable pharmaceutical products. (Cyclodextrin-based Drug Delivery, Loftsson, T., and O'Fee, R., 2002, Business Briefing: Pharmatech, p136-140).

Considerable research and development has been applied to delivery of actives to humans and economically-important animals. Actives have been formulated in numerous ways for administration, both transdermally and by ingestion. For administration by ingestion, actives have been incorporated into liposomes, granules and various types of micro-capsules. FR 2179528, U.S. Pat. No. 4,001,480, EP 0085805, GB 2162147 and EP 0242135 all describe methods/processes for the encapsulation of small molecules inside micro-organisms. In order for an active to be microbially encapsulated, it must be lipid soluble i.e. capable of permeating the lipid membrane of the microoriganism in which it is to be encapsulated. The lipophilicity of BCS Class II and IV compounds renders them ideal candidates for microbial encapsulation, and microbial encapsulation thus represents a means by which previously unformulatable Class II and IV compounds can now be formulated into usable pharmaceutical products. Examples of Class II compounds include Ketoprofen, Naproxen, Carbamezapene, and Class Iv compounds include Hydrochlorothiazide, and Furosemide.

U.S. Pat. No. 4,001,480 describes the encapsulation of actives, which are soluble within the lipid of both naturally high lipid content yeast (40-60% by weight) and yeast where the growth conditions are designed to accumulate lipid, e.g. Rihodotorula gracilis, Lipomyces species, and Endoinyces vernalis. Release of the active contained within the yeast is achieved through physical crushing or by biodegradation, for example enzymic digestion by bacteria naturally occurring within the gut.

EP 0085805 describes the use of grown high lipid content yeast (e.g. Lipomyces lipofer) and lower lipid content yeast (e.g. Candida curvata). The ability to encapsulate high concentrations of lipophilic actives in these yeast is mediated using lipid-extending substances, in which the active is dissolved. Release from the capsules is achieved by physical crushing.

FR 2179528 describes the treatment of yeast with a plasmolyser (a substance which causes contraction or shrinking of the microbial cytoplasm by exosmosis of cytoplasmic fluid), followed by infusion of a water-soluble material back into the yeast There is no description of how the active is released from the cells.

GB 2162147 describes the encapsulation of products using microorganisms containing less than 10% lipid through the use of defined organic liquid lipid-extending substances and with materials which are soluble or micro-dispersible in those substances, so that both the lipid-extending substance and the material which is soluble or micro-dispersible therein enter and are retained passively within the micro-organism. The encapsulated products are released by rupture of the capsules.

EP 0242135 describes encapsulation in yeast and other micro-organisms with a naturally low lipid content, for example brewers or bakers yeast with a lipid content of less than 10% by weight. The encapsulation process involves mixing together a liquid encapsulate, water and yeast with continuous stirring to maintain an emulsion, wherein the active diffuses into the cells. In certain examples low molecular weight solvent such as ethanol, methanol or isopropanol is used which is not retained within the cell. As with other patents the release from the capsule is due to physical crushing or from biodegradation processes.

The present inventors have discovered that the release of actives from microbial micro-capsules (yeast, fungi, bacteria, protozoa, and other unicellular organisms, including microbial derived materials which retain the cell wall structure such as that described in EP 0553176) can occur without physical breakage of the cell wall or chemical or biological degradation of the cell wall. Indeed, actives are released in a burst of activity or in a controlled manner when the external phase in which the micro-capsules are placed is a mucous membrane. Since, as has been found by the inventors, lipophilic actives are delivered upon the micro-capsules contacting a mucous membrane and without degradation of the micro-capsules, delivery of the active to a desired part of the body and absorption into the blood stream are more efficient processes. An improved efficiency means that a lower amount of the active needs to be used in any given formulation thereby reducing manufacturing costs or alternatively a greater concentration of active can be delivered directly to the membrane surface/improving uptake into the bloodstream A more efficient targeted delivery also allows for reduced exposure of the active to the harsh acid/alkaline environment of the gut/small intestine, thereby reducing chemical/biological degradation of the active, and potentially improving its efficacy. An improved rate of absorption of an active (e.g. by the mucous membranes of the stomach) means that targeting of that active to a desired part of the body is a more specific process. For example, rapid uptake of a drug in the stomach results in decreased flow through of the drug into the small intestine with a consequent reduction in non-specific targeting and waste for that active.

The micro-capsules may be employed in end-uses as a free-standing product or formulated with an excipient to facilitate delivery to a desired specific target. The use of different formulations are well known to a person skilled in the art (Remington's Pharmaceutical Sciences and US Pharmacopoeia, 1984, Mack Publishing Company, Easton, Pa., USA; United States Pharmacopoeia, ISBN: 1889788031).

According to a first aspect of the present invention, there is provided an encapsulated product comprising a plurality of micro-capsules formed from a plurality of micro-organisms and having a lipophilic active encapsulated and passively retained within said micro-capsules, said lipophilic active not being a natural constituent of said micro-organisms, said micro-capsules having:

(a) an at least substantially intact cell wall; and

(b) an intact cell membrane;

wherein said micro-capsules are formulated to target delivery of said micro-capsules and said lipophilic active to a desired at least one mucous membrane.

The term “active” as used herein is meant to include any therapeutic or otherwise active agents, i.e. a pharmaceutical compound or chemical. Illustrative categories and specific examples of actives useful in conjunction with the present invention include: anti-viral agents, analgesics, anaesthetics, anorexics, anti-arthritics, anti-depressants, anti-diabetic agents, anti-inflammatory agents, anti-parkinsonism drugs, anti-pruritics, cardiovascular drugs, anti-hypertensives, ACE inhibitors, hormones, immunosuppressives, muscle relaxants, parasympatholytics, parasympathomimetics, psychostimulants, anti-tuberculosis agents, anti-tussives, such as dextromethorphan, dextromethorphan hydrobromide, noscapine, carbetapentane citrate, and chlophedianol hydrochloride; histamine H1-receptor antagonists, such as chlorpheniramine maleate, phenindamine tartrate, pyrilamine maleate, doxylamine succinate and phenyltoloxamine citrate; histamine H2-receptor antagonists, such as ranitidine, famotidine, cimetidine, nizatidine and roxatidine; decongestants, such as phenylephrine hydrochloride, phenylpropanolamine hydrochloride, pseudoephedrine, hydrochloride ephedrine; various alkaloids, such as codeine phosphate, codeine sulphate and morphine; mineral supplements such as potassium chloride and calcium carbonates, magnesium oxide and other alkali metal and alkaline earth metal salts; laxatives, vitamins; antacids; ion exchange resins such as cholestyramine; anti-cholesterolemic and anti-lipidic agents such as gemfibrozil; anti-anrythmics such as N-acetyl-procainamide; anti-pyretics such as acetominophen, aspirin; non steroidic anti-inflammatory (NSAI) substances, and more particularly arylcarboxylic derivatives such as ibuprofen, ketoprofen, flurbiprofen, diclofenac, etodolac and naxoprene; NSAI oxicam derivatives such as piroxicam, meloxicam, tenoxicam, NSAI fenamate, indolic, and phenylbutazone derivatives; appetite suppressants such as phenylpropanolamine hydrochloride or caffeine; and expectorants such as guaifenesin. Additional useful active medicaments include coronary dilators, cerebral dilators, peripheral vasodilators, anti-infectives, psychotropics, anti-manics, stimulants, gastrointestinal sedatives and bandages, anti-diarhoeal and anti-constipation preparations, anti-anginal drugs, vasodilators, anti-hypertensive drugs, vasoconstrictors and migraine treatnents, antibiotics, tranquillisers, anti-psychotics, anti-tumour drugs, anti-coagulants, and anti-thrombotic drugs, hypnotics, sedatives, anti-emetics, anti-nauseants, anti-convulsants, neuromuscular drugs, hyper- and hypoglycaemic agents, thyroid and anti-thyroid preparations, diuretics, anti-spasmodics, uterine relaxants, nutritional additives, anti-obesity drugs, anabolic drugs, erytbropoietic drugs, anti-asthmatics, anti-histaminic or anti-cholinergic or opiate derivatives (such as codeine, dextromethorphan, ethylmorphine, noscapine, pholcodine), cough suppressants, oral mucolytics (such as acetylcisteine, ambroxol, bromhexine, carbocisteine, erdosteine, letosteine), anti-uricemic drugs and the like. Other examples of actives are well known to a person skilled in the art.

The target for delivery of the micro-capsules may be a mucous membrane.

The mucous membrane may be the membrane lining the oral cavity or buccal cavity, tongue, stomach, small intestine (duodenum or jejunum), large intestine (colon), rectum, vagina, cervix, nose, naso-pharyx, or pulmonary system (trachea, larynx, bronchi, and lungs). The mucous membrane may be the membrane lining of the digestive system of humans, domestic pets, and livestock.

The mucous membrane may be the lining of the oral cavity, buccal cavity or the tongue where the active encapsulated in micro-capsules can be for pharmaceutical use, oral health care, or as an over the counter (OTC) medicine. Drugs that can be absorbed in the mouth enter the bloodstream more rapidly and at a higher concentration than traditional swallowed tablets. The mucosal lining of the mouth is highly vascular and moves the drug directly into the heart and arterial circulation without first passing through the liver. To deliver to the mouth or tongue, the micro-capsules can be formulated as a powder, gel, spray, or tablet to treat for example, mouth ulcers, trench mouth, gingivitis or canker sores. Actives used for the treatment of e.g. mouth ulcers include choline salicyclate, lidocaine, cetalkonium chloride. Since, the bloodstream is readily accessible through the lining of the oral or buccal cavity, delivery of actives to treat non-mouth related conditions may be also be possible. Such actives may be formulated in the form of a dry or liquid (emulsion or suspension) syrup, a sachet, a chewable, a chewing gum, an orodispersible, a dispersible effervescent, a dispersible tablet, a compressed buccal tablet, a compressed sublingual tablet, a chewable tablet, and a lozenge. Chewable dosage forms for drug delivery are well known to the pharmaceutical industry.

The mucous membrane may be the membrane lining the pharynx/throat where the encapsulated product can be for pharmaceutical use or as an OTC medicine. The micro-capsules may be formulated as a compressed sweet or boiled sweet, for example as a cough sweet, where the micro-capsules may contain nonanol and/or menthol to act as a decongestant. To deliver an active to the naso-pharyngeal membranes, the micro-capsules may be formulated as a powder, gel, spray or aerosol. Analgesics and/or anaesthetics such as lidocaine and lignocaine may be encapsulated and formulated as a spray to treat tonsillitis for example.

The mucous membrane may be the membrane lining the oesophagus or stomach, where the active encapsulated in micro-capsules can be for pharmaceutical use, nutriceutical applications, or as an OTC medicine. The micro-capsules can be incorporated in a one- or two-part gelatine capsule or other similar material to aid swallowing and prevent premature release of the active in the mouth or on the surface of the tongue. For example, proton-pump inhibitors (such as Omeprazole) may be encapsulated and formulated within a gelatine capsule to treat stomach ulcers.

The mucous membrane may be the membrane lining the small/large intestine where the encapsulated active can be for pharmaceutical use or as an OTC medicine. In the small intestine release takes place both mainly due to contact with the mucous membrane but there is also some effect due to the natural emulsification system secreted in bile salts which help to emulsify the triglycerides within the micro-capsule membrane. To deliver to the small intestine, the micro-capsules may be formulated with an acid-stable enteric coating which will break down only in alkaline conditions e.g. Eudragit (Rohm and Haas), Aquacoat (FMC), and Kollicoat (BASF). There are many examples of enteric coatings, as summarized in U.S. Pat. No. 4,755,387. The use of such enteric coatings allows drugs such as Fluoxetine (Prozac) to target the small intestine. Garlic, (which contains the active ingredient alacin which is known to have beneficial effects on the cardiovascular system and can reduce cholesterol), may be encapsulated and formulated with an enteric coating, to target delivery to the small intestine, thereby eliminating the powerful odour and taste characteristics associated with other garlic preparations.

The mucous membrane may be the membrane lining the colon/rectum where the microcapsules can be for pharmaceutical use, or as an OTC medicine. Beta-glucanases produced by bacteria contained within the gut may cause release of actives prior to delivery to the colon, so specific colon-delivery agents would be required. For example, the formulation may include lactulose (which is degraded when exposed to the colon's micro-flora), so the drug is released in the colon subsequent to the formation of organic acids. The active may be prevented from degradation/absorption prior to the colon by using an outer enteric coating such as Targit (West Pharmaceuticals), and an inner cationic polymer coating for passage through the small intestine to the cecum. For example, enteric-coated peppermint oil micro-capsules can be used to treat the symptoms of Irritable Bowel Syndrome (IBS). Instead of being absorbed in the stomach and upper intestine, the enteric coating prevents release of the active until it gets to the small intestine and colon, where it relaxes the intestinal muscles. Delivery of actives to the colon or rectum can also be achieved through the use of micro-capsules formulated as a suppository, ointment, cream, or gel, for example betamethasone valerate, lignocaine and phenylphrine may be used in the treatment of haemorrhoids.

The mucous membrane may be the membrane lining the nose, where the encapsulated product can be for pharmaceutical use or as an OTC medicine. For example, actives encapsulated in microorganisms can be used for the treatment of hay fever or as a decongestant. Actives can be delivered as snuff, or as an aerosol—for example, yeast or bacterial micro-capsules containing active can be delivered to the nose via nasal applicators as an aid for introducing powdery, pharmacologically active medicaments into the nasopharyngeal space of a patient, for example in the treatment of hay-fever.

The mucous membrane may be the membrane lining the pulmonary system (i.e. larynx, trachea, bronchi, and lungs) where the active encapsulated in micro-organisms can be used for e.g. pharmaceutical use, or anti-bacterial use. Asthma may be treated through the use of encapsulated leukotriene modifiers such montelukast, zafirlukast, zileuton, or encapsulated beta agonists such as albuterol, formoterol, salmetrol, and metaproterenol. Pneumonia may be treated with encapsulated antibiotics.

The mucous membrane may be the membrane lining the vagina/cervix where the active encapsulated in micro-organisms can be for pharmaceutical use or as an OTC medicine. The micro-capsules can be formulated as a pessary, cream, ointment or gel, and may be used for prevention and treatment of thrush (e.g. using clotrimazole), as a spermicide (e.g. using nonoxynol-9), an anti-inflammatory agent, an anti-bacterial agent (e.g. such as benzylalkonium chloride), or as an anti-cancer agent.

The mucous membrane may be the membrane lining the digestive system of humans, domestic pets, and livestock, where partial release of the active takes place throughout the digestive system For example, delayed and controlled release of an active can take place when the active is released throughout the entire digestive system of humans. The biological membrane in this instance is the tunica mucosa, which lines the upper gastrointestinal tract, stomach, small intestine and colon.

Accordingly, the micro-capsules may be formulated as a dry or liquid (emulsion or suspension) syrup, a sachet, a chewable, a chewing gum, an orodispersible, a dispersible effervescent, a dispersible tablet, a compressed buccal tablet, a compressed sublingual tablet, a chewable tablet, a melt-in-the-mouth, a lozenge, a paste, a powder, a gel, a tablet, a compressed sweet, a boiled sweet, a cream, a suppository, a snuff, a spray, an aerosol, a pessary, or an ointment.

According to a second aspect of the present invention there is provided a method of manufacture of an encapsulated product, wherein said encapsulated product comprises a plurality of micro-capsules formed from a plurality of micro-organisms, comprising the step of

(i) contacting said micro-organisms with a lipophilic active to encapsulate said lipophilic active within said micro-organisms;

said lipophilic active being encapsulated and passively retained within said micro-capsules,

said lipophilic active not being a natural constituent of said microorganism, said micro-capsules having;

(a) an at least substantially intact cell wall; and

(b) an intact cell membrane, further comprising the step of:

(ii) formulating said micro-capsules to target delivery of said micro-capsules and said lipophilic active to a desired at least one biological membrane.

Various methods of encapsulation are known and include those described in FR 2179528, U.S. Pat. No. 4,001,480, EP 0085805, GB 2162147 and EP 0242135.

The method of encapsulation of a lipid soluble active may utilise micro-organisms which are grown in conditions which promote accumulation of lipid within the cell. By increasing the cellular lipid content (e.g. to 40-60%), greater quantities of a lipid soluble active may be stored within the cell. The active may be contacted with the micro-organism and incubated for a desired period of time to encapsulate it, and the resulting encapsulated substance may be harvested After encapsulation and harvesting, the micro organism may be treated with a proteolytic enzyme in order to soften the micro-capsules. This softening treatment may also be performed prior to encapsulation of the lipid soluble active. More efficient encapsulation may be attained through heating during the encapsulation process, or through the application of physical pressure to the micro-organism/active mixture.

The microorganism may have a natural lipid content ranging from less than 10% to greater than 50%. Alternatively, growth media may be employed which promote the storage of lipid within the microbial cell, thereby increasing the lipid content to values greater than the natural lipid content.

Prior to encapsulation, a plamolyser may be employed. This substance causes contraction or shrinking of the microbial cytoplasm by exosmosis of cytoplasmic fluid.

The micro-organism is in grown form, i.e. it has been harvested from its culture medium after a period of growth, and it is intact and not lysed. Preferably, the micro-organism is alive at the commencement of the encapsulation process since more efficient encapsulation is usually achieved, however a micro-organism which has been subjected to conditions such as irradiation (to destroy its ability to propagate), micro waving (for sterility purposes) or spray drying may also be employed.

The active should be in liquid form or in a solution during the encapsulation process. The active may be a liquid (including oil) in its normal state, or it may be a solid, in which case it should be dissolved or micro-dispersed in a solvent which is lipid soluble. Suitable solvents include:

(a) primary alcohols within the range C4 to C12, such as nonanol and decanol (higher alcohols containing a linear chain of more than twelve carbon atoms are too large for encapsulation);

(b) secondary and tertiary alcohols;

(c) glycols such as diethylene glycol;

(d) esters—any ester where the straight carbon chain is greater than 2 and less than or equal to 12, e.g ethyl butyrate, triacetin;

(e) aromatic hydrocarbons such as xylene, and acetophenone,

(f) any aromatic lipophilic oil with no straight chain branch greater than 12 carbons.

(g) carboxylic acids between C3 and C12.

Alternatively a solid active may be encapsulated e.g. menthol, however it must be lipophilic to encapsulate successfully and it should be soluble in one of the above solvents or melt below 80° C. Prolonged temperatures above 80° C. would damage the cell membrane beyond repair. Ideally for the process the active should be liquid between 40 and 65° C. since higher temperatures may result in degradation of the active.

Multiple actives may be co-encapsulated—e.g. caffeine and aspirin/paracetamol for treatment of a common cold, or influenza.

Methanol, ethanol and isopropanol are very low molecular weight volatile solvents, which can be used to assist in encapsulation but do not actually encapsulate themselves. If used to encapsulate a material the active must be soluble in e.g. ethanol and when added to e.g. 3 or 4 parts water the active must stay in solution. There must always be some water present to swell the yeast thereby hydrating the membrane, or encapsulation will not take place. The ethanol evaporates during the process and the active, which must be at least partially soluble within the yeast membrane, is encapsulated. Residual ethanol will evaporate during post-encapsulation treatments such as spray drying.

Several criteria must be considered in order to predict whether an active can be encapsulated. Actives having a benzene or naphthalene ring appear to be particularly suitable for encapsulation. Actives with an octanol/water partition coefficient (logP) of between 0.5 and 4.0 will encapsulate well. Molecular weight must also be considered—actives with a molecular mass less than 1000 Daltons can generally be encapsulated. Size is also important—since straight chain hydrocarbons greater than C12 do not encapsulate, any molecule containing a straight chain C12 stretch or greater will not encapsulate, nor will a molecule with a rigid structure similar in length to a C12 chain. Molecules with a greater number of carbons than C12 can be encapsulated as long as the structure contains benzene rings, e.g. phenolics, or naphthalene rings, etc. Molecules (actives) with a small molecular diameter work best Volatile molecules with one to three carbons do not encapsulate, e.g. ethane, ethanol, propanol, whereas molecules containing four or more carbon atoms generally do encapsulate. The range for encapsulation in terms of straight chain carbon atoms lies between C4-C12. Beyond these criteria, the suitability of actives for encapsulation may be found by a simple trial of the method of the invention.

The encapsulation treatment may be performed at normal ambient temperatures but preferably the temperature is elevated, in order to expedite the encapsulation treatment. A suitable elevated temperature may be in the range 35 to 60° C.

The encapsulation treatment preferably comprises mixing the micro-organism with the active in a liquid medium, especially an aqueous medium, to attain good dispersion and contact of the micro-organism with the active.

The encapsulation treatment may be continued until optimum encapsulation has been achieved. Encapsulation may usually be observed microscopically as one or more globules of the active contained within the microbial cell, unless the yeast is grown in a harsh environment (such as high alcohol content), in which case the cell wall can be thickened which makes direct visualisation by light microscopy more difficult. In such instances, transmission electron microscopy (TEM) may be required. The encapsulation treatment may take a few hours before the optimum level of encapsulation is achieved.

After encapsulation, residual low molecular weight solvents such as ethanol, methanol and propanol may be removed after the encapsulation process by evaporation or other air drying processes. Drying by evaporation in inert gases or oxygen free atmospheres can also aid the process where sensitive actives are used. Water may also be removed by spray- or freeze-drying. Water may also be removed by evaporation by putting the micro-capsule suspension in a dry oven.

A pretreatment bleaching step may be carried out prior to encapsulation. For example, the treatment may be performed at room temperature for up to one hour where the micro-organism is treated with a solution of an alkaline bleach solution comprising 0.2 M sodium hydroxide/1% w/v hydrogen peroxide, with a pH value of between 9-10. Sodium silicate may be added to the mixture as an anti-foam agent. The resulting micro-organisms are generally off-white in colour, and the cell well may be more porous. For example, in the case of bleached yeast, the cells when dry may absorb between 5-10 times their weight in water, compared to untreated yeast cells which may absorb between 2-3 times their weight in water. This increased capacity of the bleached yeast to absorb water means that encapsulation is usually performed in a greater volume of liquid, thereby avoiding problems associated with increased viscosity.

Prior to, or in some cases during the encapsulation process, the micro-organism may be treated at an elevated temperature and/or with an enzyme and/or with a chemical such as sodium hydroxide or a magnesium salt to improve the efficiency of encapsulation. Enzymes such as pepsin, trypsin, chymotrypsin, chitinase, b-glucanase serve to degrade the microbial cell wall. Sodium hydroxide or magnesium salts enhance permeability of the micro-organism. The micro-organism may then be mixed with the active to be encapsulated and incubated until optimum encapsulation is achieved (as determined by light or electron microscopic analysis of the micro-capsules). High shear mixing may be used to aid dispersion of the yeast and improve the contact between the yeast and active, aiding encapsulation.

After encapsulation of the active a conditioning treatment of the resulting micro-capsules may be performed to remove colour, taste and odour of the microbial micro-capsules. This conditioning treatment comprises incubating the micro-capsules in a dry environment such as an oven or heat chamber at room temperature for several weeks or months, or at an elevated temperature of up to 40° C. for hours/days.

In the case of yeast, the encapsulation process results in the accumulation of actives within the naturally double walled capsule. Yeast cell walls are generally 80-90% polysaccharide, including predominant glucans such as 1,3-β-glucan, and also the long chain carbohydrate polymer chitin which adds rigidity and structural support to the cells. Proteins (such as mannoproteins), lipids and polyphosphates together with inorganic ions make up the cell wall cementing matrix. The inner membrane is a typical lipid bilayer. The yeast cell wall, unlike many food grade capsules, is insoluble and therefore the micro-capsules can be wet and dry processed. When the yeast microcapsules are spray dried a free flowing powder is produced made up of agglomerated particles comprising numerous yeast cells. Depending on drying conditions the dry particle size can range between 10 and 300 microns. For large particles a fluidised bed is required. The product can also be prepared as a cake, suspension, produced by pressing, or rotary drying. Particle size or a mixture of particle sizes may be useful to control release rates.

The micro-capsules may be washed after encapsulation to remove residual unencapsulated material and isolated by centrifuging, freeze-drying or spray-drying.

The micro-organism is preferably a fungus. Typical fungi are yeasts e.g. Saccharomyces cerevisiae (brewer's yeast and baker's yeast), Kluyveromyces fragilis (dairy yeast) and Candida utilis. Yeasts may be selected from the taxonomic order Endomycetales. The micro-organism may be a filamentous fungus, e.g. Aspergillus niger. The spore, mycelium and giant cell forms of filamentous fingi may be employed. The micro-organism may be a mold, e.g. Fusarium graminearium. Other micro-organisms which may be employed are bacteria and algae. Any relatively large protozoa also may be utilised—examples of such protozoa include Bacteriodes succinogenes, Etidinium ecazidatum, Entodinium caudalum, Eudipolodinium neglectum, Eudiplodinium maggii, Diplodinium dentation, and Polyplastron multivesiculatum.

According to a third aspect of the present invention there is provided a method of treatment of a patient comprising administering to said patient a medicament comprising the encapsulated product according to the first aspect of the present invention i.e. substantially intact micro-capsules containing a lipophilic active so that said intact micro-capsules contact a mucous membrane of said patient, wherein said micro-capsules are coated with a formulation to target delivery of said active to a desired part of the body. Naturally, the patient may be in need of treatment with said medicament

The invention will be further apparent from the following description and figures, which show, by way of example only, forms of targeted delivery, in which:—

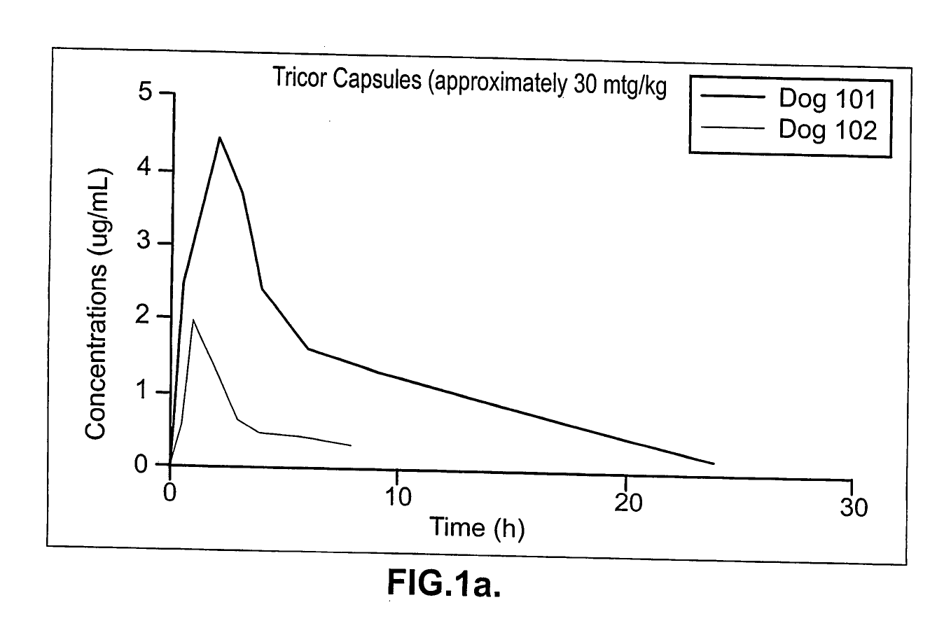

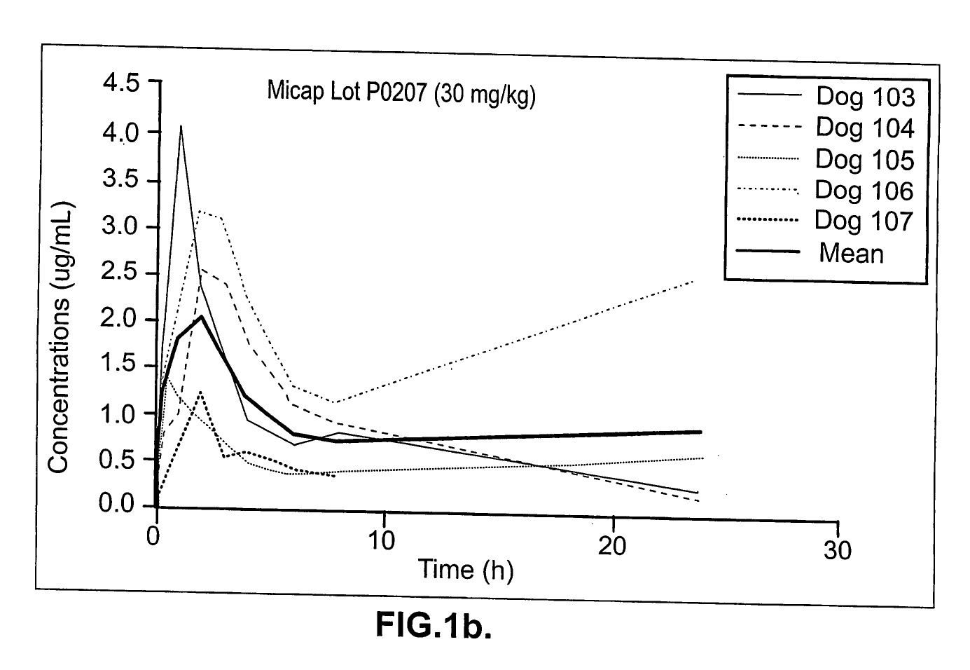

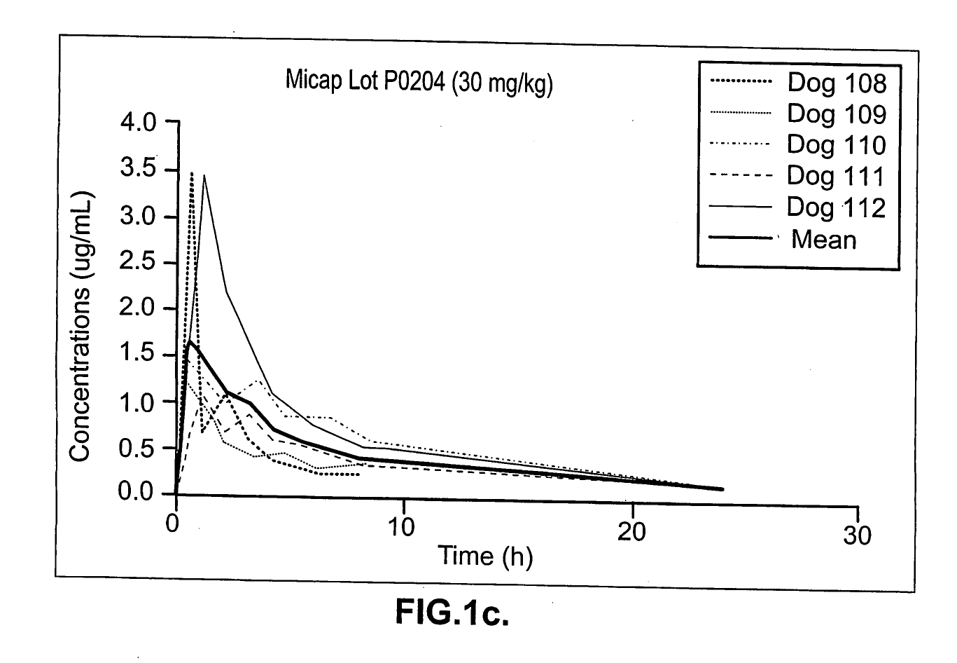

FIGS. 1a, b and c illustrate individual and mean test compound plasma concentration-time profiles; and

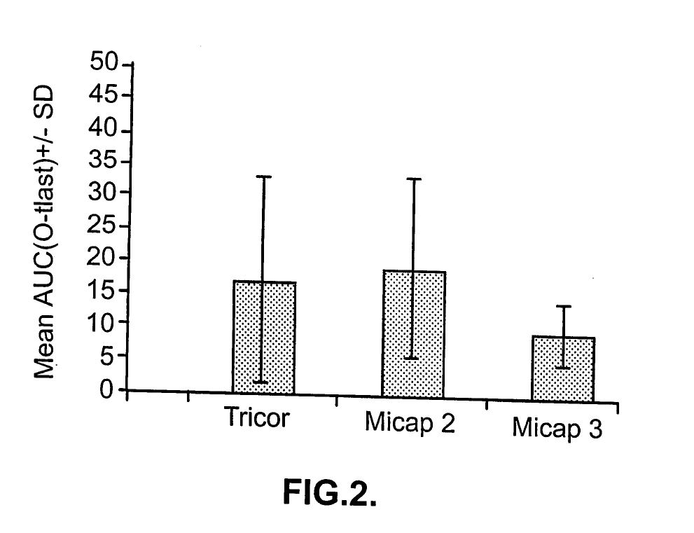

FIG. 2 illustrates statistical analysis of a comparative study.

### EXPERIMENTS

The following examples detail the production and formulation of various encapsulated products. Additionally, the methods employed to encapsulate actives are also described

### Example 1

The yeast Saccharomyces cerevisiae was maintained on MYGP agar slopes (0.3% (w/v) each of malt extract and yeast extract, 0.5% bacterial peptone, 2% (w/v) glucose; 2% (w/v) agar). A loop of yeast was transferred aseptically to 10 ml MYGP broth, (media prepared as above but without agar) and incubated overnight at 30° C. The broth was aseptically transferred to a fermenter containing 5-litres working volume of MYGP broth. The culture was incubated for 3 days at 30° C. and the yeast harvested by centrifugation using a MSE Mistral 3000i centrifuge (2000×g). The harvested yeast was washed with water to remove excess media and suspended in water to a final solids content of 33% w/v in a jacketed glass vessel at a temperature of 55° C. The yeast was agitated with top siring using a Teflon paddle (Stuart Scientific SS10), at approximately 300 rpm. Pre-melted menthol was added to the mixture to half the dry weight of the yeast and the mixture stirred continuously for a further 5 hours. The yeast cells containing menthol were then removed by centrifugation, washed with warm water and dried by spray-drying. The resulting yeast capsules contained crystals of menthol, at 33% w/w.

The menthol micro-capsules were incorporated in a tablet using conventional methods known to those in the pharmaceutical industry, which when placed in the mouth released the flavour and odour of menthol on contact with the mucous membrane. More menthol was released as the tablet dispersed in the mouth by the action of saliva, providing a prolonged, decongestant effect.

### Example 2

Yeast, Saccharomyces cerevisiae (62F) was obtained from William Bioenergy as a spray dried powder, this yeast was light in colour and had little yeast flavour due to the chosen culture media, which was based on corn syrup. The dry powder washed with water to remove excess media components and the resultant yeast, approximately 65% of the dry weight of the spray dried powder, was suspended in water to a final solids content of 35% w/v in a jacketed glass vessel, temperature 42° C. The yeast was agitated with top stirring, Stuart Scientific SS10, with Teflon paddle, at approximately 300 rpm. Ibuprofen dissolved in triacetin (10% w/w) was added to the mixture to approximately half the dry weight of the washed yeast and the mixture stirred continuously for 6 hours. The yeast cells containing triacetin and ibuprofen were then removed by centrifugation, washed with warm water and dried by spray-drying. The resulting yeast capsules contained 36% w/w triacetin and 3.7% w/w ibuprofen.

The resulting powder was placed in a two-part gelatin capsule and could be used to deliver ibuprofen directly to the stomach lining allowing speedier uptake and faster pain relief.

### Example 3

Commercially available dry bakers yeast (300 g) (Saccharomyces cerevisiae) was suspended in one litre of a 0.2 M solution of sodium hydroxide in water containing 40 g per litre of sodium silicate. Hydrogen peroxide was added until the concentration reached 1% w/w and the resulting suspension was then gently stirred for one hour at room temperature. The yeast was then removed by centrifugation, washed with water to remove excess bleaching agent and dried by spray drying. The yeast produced was white to off-white in colour and in suspension had a creamy texture with no discernible yeasty odour.

The spray-dried material was stored dry at room temperature ready for future encapsulation processes.

A portion of the suspension before drying, was adjusted to 20% solids with water. The viscosity of the bleached and deodorised yeast was too great to obtain the desired emulsion characteristics using a similar concentration as the unbleached yeast. The bleached and deodorised yeast suspension was stirred using a rotary stirrer at 350 rpm for 4 hours at 44° C. in the presence of loratidine dissolved in terpene oil at a concentration of 15% w/v, (loratidine/terpene oil mixture was added to approximately 50% of the weight of dry yeast). The yeast cells containing Loratidine in terpene oil were then removed by centrifugation, washed with water and dried by spray-drying. The dry product contained approximately 24% terpene oil and 3.3% Loratidine.

The powder can be applied in a measured dose powder applicator to the nasal membranes giving relief from hay-fever symptoms.

### Example 4

Yeast grown in media based on corn syrup as described in example 2 were bleached and deodorised using the procedure described in example 3 and the resulting yeast were suspended in water to approximately 20% solids. Tea tree oil was added to the yeast suspension, whilst stirring using a rotary stirrer at 350 rpm. The mixture was agitated at 60° C. until the concentration of tea tree oil within the yeast did not increase. The yeast cells containing tea tree oil were then removed by centrifugation, washed with water and dried by spray-drying. The powder containing 45% tea tree oil by weight was then formulated into a chewable tablet. The yeast micro-capsules within the tablet released the tea tree oil when in contact with the mucous membranes in the mouth in the presence of moisture, (both essential for tea tree oil release), delivering a natural antibacterial effect in a sustained and prolonged manner.

### Example 5

Bleached and deodorised Torula yeast (Candida utilis) was suspended in water at approximately 18% solids. Omeprazole dissolved in nonanol to a final concentration of 8% w/w was added to the yeast suspension, whilst stirring using a rotary stirrer at 320 rpm; the nonanol/Omeprazole mixture was added to a final concentration of approximately 40% to that of the dry Yeast The mixture was stirred continuously for 8 hours at 40° C. After incubation the yeast cells were harvested by centrifugation and washed twice with water. The yeast pellet was frozen at −20° C. and dried by freeze-drying for 24-48 hours. The resulting dry cake was milled such that 100% of the particles were less than 100 microns in diameter. Upon analysis, the dry capsules were determined to contain approximately 26% nonanol and 2% omeprazole.

The capsules were formulated into a hard gelatine capsule ready to use in the treatment of gastric ulcers.

### Example 6

Bleached and deodorised Kluyveromyces fragilis was suspended in water to approximately 23% solids. Chloramphenicol dissolved in triacetin to a final concentration of 10% w/v was added to the yeast suspension, whilst stirring using a rotary stirrer at 320 rpm; the triacetin/chloramphenicol mixture was added to a final concentration of approximately 50% that of the dry yeast. The mixture was stirred continuously for 6 hours at 50° C. After incubation the yeast cells were harvested by centrifugation and washed twice with water. The yeast cell containing triacetin at 32% w/w and chloramphenicol at 3% w/w were diluted to 25% solids and dried by spray drying.

The chloramphenicol micro-capsules were incorporated in a tablet using conventional methods known to those in the pharmaceutical industry, which were then swallowed releasing their contents onto the surface of the digestive tract.

### Example 7

Aspergillus oryzae cellular mrass obtained from the citric acid industry was washed with water, then bleached as in example 3, producing white to off-white mycelial strands with intact cell walls and cellular membranes. The mass was placed in a 2-litre rotary shaker at 30% solids and 50% of the weight of fungal mycelial of a bergemot and manuka oil mixture added. The mycelial mass/essential oil mixture was shaken continually in a closed vessel for 3 hours at 60° C. The fungal mycelia containing the essential oil mixture were removed by filtration through a wire mesh, 100 microns. The mycelia were resuspended in water (10 g/2 L water) and paper-like sheets were cast using conventional test-paper making equipment In some cases an additional porous carrier sheet was required to ensure easy removal of the wet fungal mycelia from the paper-making grid. A portion of the material was re-suspended in water to a final solid concentration of 65%. This material was placed in a mould approximately 9 cm in diameter, and a depth of 1 cm and frozen at −20° C. overnight; the frozen pad was then dried by freeze-drying. In both formats the essential oil content was 19% manuka oil and 18% bergamot oil by weight. The paper like material or pad when placed in the mouth, on or under the gum, released the essential oils, producing an antibacterial and anti-inflammatory effect, useful in the treatment of mouth ulcers and bacterial infection.

### Example 8

Bleached and deodorised Saccharomyces cerevisiae were suspended in water to approximately 20% solids and Ketoprofen (20% w/v) dissolved in menthone (at 55° C.) was added to a final concentration equal to 50% the weight of the dry yeast The mixture was stirred at 65° C. for 1 hour at which point the yeast cells containing encapsulated ketoprofen in menthone were harvested by centrifugation.

The ketoprofen micro-capsules were incorporated in a tablet using conventional methods known to those in the pharmaceutical industry, and the tablets when swallowed released their contents onto the surface of the digestive tract.

### Example 9

Saccharomyces cerevisiae (62F) cell (obtained from William Bioenergy) were pre-processed by washing with water to remove, excess media and yeast extract components such as simple and complex carbohydrates (single and multi glucose units), glycerol, acetic and lactic acid (the fermentation media), herein defined as defined as non-yeast. The processed yeast (300 kg dry powder) was mixed with 700 L water in a 1,500 L stainless steel jacketed, round bottom vessel. The mixing could take a number of forms as follows:

The yeast were added over a 20 minute period while the tank was agitated with a high shear Silverson mixer. Homogenous dispersion took approximately 25 minutes.

Alternatively the yeast and water were added and dispersed in the tank using a vortex dispersion (wetting system) placed over the tank. The mixture was then agitated using a slow paddle stirrer or a marine prop mixer (Lightnin). Homogenous dispersion took 10 minutes and agitation was continued until the separation stage.

In another alternative mixing regime, the yeast and water were added to the tank and dispersed by adding the yeast to the water, then pumping the material in a closed return system through an in-line mixer. Homogenous dispersion took 20 minutes and agitation was continued until the separation stage.

Once a homogenous dispersion was achieved the mixture was continually agitated at 40° C. for 30 minutes. The use of hot water at 60-80° C. washed out 40% of the non-yeast fraction in the first wash. Once the material had been washed for 30 minutes it was passed through a Westfalia 300 decanter at 10 L/min. After washing, 90 kg (non-yeast) of the 300 kg yeast powder was removed, leaving 210 kg of yeast cells. For encapsulation the yeast was passed through the separator and into the encapsulation vessel. The concentration of the yeast at this stage could be as high as 65% solids. Water was added to the encapsulation vessel to dilute the yeast to approximately 30% solids.

As an alternative to encapsulation at this stage, further washing processes can take place. Repeating the washing process removed a-further 8% non-yeast and for a second time a further 5% was removed. Care was taken to avoid excess washing which can damage the yeast causing the cells to agglomerate, making downstream processing very difficult. To the 30% solids yeast suspension containing 210 kg of yeast cells, 100 kg of an equal mixture of manuka oil, tea tree oil and lemon grass oil was added and incubated with stirring at 45° C. for 2 hours. The cells were concentrated using the Westfalia decanter to a solid content of 42-45% and dried by spray drying. To help recover more encapsulated product a Westfalia SAI (self cleaning centrifuge) was used. In this case a further 10% encapsulated essential oil mixture was recovered.

The standard product manufactured was a powder with a 30 micron particle diameter, (90% of the particles fell within this range). Alternatively a two fluid nozzle was used to dry the product giving particles evenly distributed in the range of 30-90 microns. In another format more pressure was used to spin the atomiser faster, (35,000 rpm) in this case the particle size of the encapsulated essential oil mixture was reduced to 20 microns. The yeast capsules contained approximately, 12% manuka oil, 10% tea tree oil and 13% lemon grass oil by weight.

A portion of the yeast encapsulated essential oil when spray dried was blown through a secondary cyclone to cool the product before it was packaged. This reduced any “yeasty” or “musty” odours that can become associated with the product.

A further portion was conditioned by blowing across a bed of cool dry air and collecting the product in a cooled cyclone. The addition of an inert gas, such as nitrogen, into the head space of the packaged final material also cut down on any unwanted spoilage/odours.

As an alternative a portion of the encapsulated slurry was further processed before drying. As the essential oil enters the yeast cell, further yeast extract was produced and to ensure that no essential oil residue remained on the surface of the yeast cells the yeast capsules were washed counter currently, using 2 separators, (for ultrapure samples up to 5 separators were used) fed by in-line mixers. The wash water is reused in each of the washing steps and concentrates the washings. The yeast was pumped to a second tank where the yeast was diluted by 50% using mains water. The yeast slurry was then agitated using a slow paddle mixer before being pumped into an NA7 Westfalia separator at 7 L/min (the feed was passed through an in-line brush strainer to remove any large particles that may have blocked the nozzles in the centrifuges). The feed to the first NA7 was at approximately 15% solids and left the first separator at 20% solids where it was fed into a second separator (the separators were running on 4×0.5 mm nozzles). The second separator was fed via an inline mixer, which diluted and washed the yeast to 15% solids. This was in turn concentrated using the decanter centrifuge to 42-45% solids and spray dried or was concentrated further to approximately 62% solids and processed as a cake.

A portion of the concentrated yeast containing encapsulated essential oil was dried over a fluidised bed producing agglomerates of yeast capsules in the range 300-500 microns.

In a product which had been packaged without any pre or post treatment to reduce ‘yeast’ odour' after 3 months in dark and cool conditions the “yeasty” notes had disappeared; this process was facilitated by rotating the package on a weekly basis. The micro-capsules were used in anti-bacterial and anti-fungal applications.

### Example 10

Tricor (RTM fenofibrate) capsules (obtainable from Abbott Laboratories) were compared for bioavailability with fenofibrate encapsulated within Williams yeast (Saccharomyces cerevisiae (William's yeast, available from Aventine Renewable Energy Co. Inc.) in accordance with the present invention.
Formulation 1: Fenofibrate control (Tricor capsules, marketed micronized fenofibrate from Abbott Laboratories in 67 and 200 mg capsules)
Formulation 2: Micap 2 (135 mg per g Williams yeast), Lot P0207
Formulation 3: Micap 3 (180 mg per g Medical yeast), Lot P0204
   Dosage preparation for the Micap 2 formulation (135 mg per g) was prepared by mixing with water to a final concentration of 30 mg fenofibrate/mL. For example to prepare 60 mLs of final formulation (Micap 2 in water), weight 13.3 g of Micap 2(135 mg fenofibrate/g Micap 2) and q.s. to 60 mL with water. The suspension was dosed at 1 mL/kg of animal body weight (30 mg/kg). The test system used comprised a dog model, using a purebred Beagle since this is a universally used model for evaluating toxicity of various classes of chemicals.
   Dosage preparation for Micap 3 (180 mg per g) was done by mixing with water to a final concentration of 30 mg fenofibrate/mL. For example to prepare 60 mLs of final formulation (Micap 3 in water), 10 g of Micap 3 was weighed (180 mg fenofibrate/g Micap 3) and q.s to mL with water. The suspension was dosed at 1 mL/kg (30 mg/kg).

The test animals were at least 5 months of age and weighted approximately 9.0 to 12.0 Kg.

| | | | | NUMBER OF ANIMALS | |
|---|---|---|---|---|---|
| | | | | | PHARMACO- |
| | | | | | KINETIC |
| | TREAT- | Dose | Dose | DOSING | SAMPLING^{a} |
| GROUP | MENT | (mg/kg) | (mL/kg) | M | M |
| 1 | Fenofibrate | 30 | Na^{b} | 2 | 2 |
| | capsules | | | | |
| | (Tricor) | | | | |
| 2 | Micap 2 | 30 | 1^{c} | 5 | 5 |
| 3 | Micap 3 | 30 | 1^{c} | 5 | 5 |

^{a}PK samples were collected from each animal. Samples were be collected predose and at 0.5, 1, 2, 3, 4, 6, 8 and 24 hours after dosing.
^{b}Estimated number of 200 mg and 67 mg capsules needed to achieve 30 mg/kg.
^{c}Based on 30 mg/mL fenofibrate in Micap suspension.

The route of administration for group 1 was orally via capsules while for group 2 and 3 administration was via the duodenum using an endoscope. The animals in group 2 and 3 were anaesthetized with Telazol 5 mg/kg IM, or less. Isoflurane via a vaporizer delivered in O₂ with a nose cone was used as needed. An endotracheal tube was placed and general anesthesia maintained with Isoflurane in O₂. A flexible endoscope was passed down the esophagus through the stomach into the duodenum. A catheter was subsequently passed down the working channel and the test article delivered. The catheter was then flushed with 3 ml of a tap water rinse and endoscope and catheter withdrawn. The endotracheal tube was removed when the animal regained its swallowing reflex and the animal was monitored for normal recovery from anesthesia. It will be appreciated that the oral route is an accurate means of delivering the test article to provide a pharmacokinetic assessment of the marketed fenofibrate, Tricor (RT). Duodenal delivery for the Micap formulation was necessary to avoid breakdown in the stomach.

The test compositions were administered once, on Day 1 at a dosage of 30 mg per Kg of body weight of test animal.

Composition Administration

For all three groups the test animals were fasted for at least 18 hours prior to test composition administration.

Pharmacokinetic Samples

Blood samples will be collected (approximately 2 mL whole blood) via the jugular vein or other appropriate vessel. Samples will be placed in tubes containing EDTA and stored on an ice block until centrifuged. Plasma was separated and frozen at approximately −70° C. within 60 minutes of collection. The samples were then analysed for serum concentrations of the test composition.

Subsequently, quantitative determination of fenofibric acid in dog plasma with EDTA was carried out. A calibration curve in dog plasma spiked with fenofibric acid was prepared so that a linear range of detection of 0.100 μg/mL to 10.0 μg/in L was achieved. The assay utilized a protien precipitation with methanol. Following vortexing and centrifuging, the supernatent was removed and directly injected onto the HPLC

Chromatographic Conditions

| | |
|---|---|
| Instrument | |
| Controller: | Waters Milliennium³² Data Colleciton |
| | Software (ver 4.0) |
| Pump: | Waters 2695 HPLC pump |
| Automsampler: | Waters 3695 Autosampler |
| Detector: | UV at 287 nm |
| Column: | Phenomenex luna C-18, 250 mm × 4.6 mm, |
| | 5 μm |
| Guard Column: | None |
| Column Temp: | Ambient |
| Mobile Phase: | 55:45 ACN: 0.2% Phosphoric acid solution (v/v) |
| Flow Rate: | 1.2 mL per minute |
| Injection Volume: | 20 μL |
| Operation: | The chromatography system was |
| | equilibrated with mobile phase for |
| | approximately 60 minutes. |
| Peak Parameters: | Approximate Fenofibric acid Retention |
| | Time: ˜11.5 min. |
| Reagents | |
| Acetonitrile (ACN) | HPLC Grade |
| Deionized (DI) Water | KAR Laboratories, Inc. Reverse |
| | Osmosis System |
| Menthanol | HPLC Grade |
| Phosphoric Acid | HPLC Grade |

Sample Preparation
1. 100 μL of sample was pipetted into a 2.0 mL microcentrifuge tube.
2. 20 μL of methanol was added.
   3. Hand shake briefly.
4. 500 μL of precipitation solution (methanol) was added and the sample vortexed for ˜20 minutes
5. Centrifuge at ˜14,000 rpm for ˜ minutes
6. The supernatant was transfered directly to an HPLC vial.

Waters HPLC instrument software was used to directly back-calculate concentrations from peak heights based on a eight point linear curve with 1/concentration weighing where Y=mX+b (Y=peak height, m slope, X concentration and b intercept).

Dosing

| | | | | | | | Nom. |
|---|---|---|---|---|---|---|---|
| Animal | | | | Capsules | Suspension | Conc. | Dose |
| No. | Group | BW (kg) | Treatment | (mg) | (mL) | (mg/mL) | (mg/kg) |
| 101 | 1 | 9.00 | Tricor | 267 | — | — | 29.7 |
| 102 | 1 | 10.25 | Tricor | 333 | — | — | 32.5 |
| 103 | 2 | 10.85 | William's Yeast | — | 10.9 | 30 | 30.1 |
| 104 | 2 | 10.70 | William's Yeast | — | 10.7 | 30 | 30.0 |
| 105 | 2 | 10.30 | William's Yeast | — | 10.3 | 30 | 30.0 |
| 106 | 2 | 9.55 | William's Yeast | — | 9.6 | 30 | 30.2 |
| 107 | 2 | 9.40 | William's Yeast | — | 9.4 | 30 | 30.0 |
| 108 | 3 | 8.95 | Medical Yeast | — | 9 | 30 | 30.2 |
| 109 | 3 | 10.15 | Medical Yeast | — | 10.2 | 30 | 30.1 |
| 110 | 3 | 10.05 | Medical Yeast | — | 10.1 | 30 | 30.1 |
| 111 | 3 | 10.50 | Medical Yeast | — | 10.5 | 30 | 30.0 |
| 112 | 3 | 11.00 | Medical Yeast | — | 11 | 30 | 30.0 |

William's Yeast = 135 mg fenofibrate/g (Micap 2) mixed with water to yield 30 mg/mL
Medical Yeast = 180 mg fenofibrate/g (Micap 3) mixed with water to yield 30 mg/mL

Individual and mean plasma concentrations of fenofibric acid are listed hereinabove.

Individual and mean fenofibric acid plasma concentration-time profiles are presented by treatment group in FIGS. 1a,b and c.

Results from pharmacokinetic analysis are presented in the following table.

Individual and mean plasma concentrations of fenofibric acid

| Collection Dates: | | | | Non-GLP Analysis of Fenofribric Acid in Dog Plasma | | | | | |
|---|---|---|---|---|---|---|---|---|---|
| Apr. 3, 2003 & Apr. 4, 2003 | | | | KAR ID: 031737 | | | | | |
| Units = ug/mL | | | | Gender = Male | | Day = 1 | | | |
| Animal N^{o} | 0 Hour | 0.5 Hour | 1 Hour | 2 Hour | 3 Hour | 4 Hour | 6 Hour | 8hour | 24 Hour |
| | TRICOR | | | | | | | | |
| 101 | <0.100 | 2.50 | 3.01 | 4.44 | 3.73 | 2.37 | 1.58 | 1.37 | 0.156 |
| 102 | <0.100 | 0.562 | 1.96 | 1.21 | 0.608 | 0.433 | 0.400 | 0.310 | <0.100 |
| | Micap 2 | | | | | | | | |
| 103 | <0.100 | 2.04 | 4.11 | 2.33 | 1.56 | 0.906 | 0.663 | 0.761 | 0.207 |
| 104 | <0.100 | 0.759 | 0.938 | 2.51 | 2.37 | 1.70 | 1.07 | 0.872 | 0.161 |
| 105 | <0.100 | 1.30 | 0.965 | 0.733 | 0.568 | 0.442 | 0.316 | 0.336 | 0.568 |
| 106 | <0.100 | 1.58 | 2.25 | 3.30 | 3.03 | 2.19 | 1.26 | 1.10 | 2.53 |
| 107 | <0.100 | 0.302 | 0.603 | 1.17 | 0.505 | 0.572 | 0.392 | 0.319 | <0.100 |
| Mean | <0.100 | 1.1962 | 1.7732 | 2.0086 | 1.6066 | 1.162 | 0.7402 | 0.6776 | 0.8665 |
| | Micap 3 | | | | | | | | |
| 108 | <0.100 | 3.46 | 0.653 | 1.10 | 0.608 | 0.390 | 0.244 | 0.262 | <0.100 |
| 109 | <0.100 | 1.28 | 1.08 | 0.665 | 0.489 | 0.500 | 0.291 | 0.346 | <0.100 |
| 110 | <0.100 | 1.45 | 1.29 | 1.06 | 1.27 | 0.948 | 0.867 | 0.623 | 0.138 |
| 111 | <0.100 | 0.635 | 1.11 | 0.682 | 0.873 | 0.619 | 0.517 | 0.318 | 0.167 |
| 112 | <0.100 | 1.65 | 3.39 | 2.21 | 1.71 | 1.09 | 0.753 | 0.541 | 0.154 |
| Mean | <0.100 | 1.695 | 1.5046 | 1.1434 | 0.99 | 0.7094 | 0.5344 | 0.418 | 0.153 |

Pharmacokinetic Analysis

Results from PK analysis are presented in the following table.

| | | Tmax | Cmax | AUC₍₀₋ₜₗₐₛₜ₎ | Kₑₗᵢₘ | T½ |
|---|---|---|---|---|---|---|
| Dog n^{o} | Formulation | (h) | (ug/mL) | (h * ug/mL) | (1/h) | (h) |
| 101 | Tricor | 2 | 4.44 | 28.6 | 0.134 | 5.19 |
| 102 | Tricor | 1 | 1.96 | 5.26 | 0.118 | 5.87 |
| N | | 2 | 2 | 2 | 2 | 2 |
| Mean | | 1.5 | 3.2 | 16.9 | 0.126 | 5.53 |
| SD | | — | 1.75 | 16.5 | 0.011 | 0.481 |
| 103 | Micap2 | 1 | 4.11 | 18.1 | 0.0721 | 9.62 |
| 104 | Micap2 | 2 | 2.51 | 18.2 | 0.105 | 6.58 |
| 105 | Micap2 | 0.5 | 1.3 | 11.4 | n.d | n.d |
| 106 | Micap2 | 2 | 3.3 | 43.1 | n.d | n.d |
| 107 | Micap2 | 2 | 1.17 | 4.18 | 0.146 | 4.75 |
| N | | 5 | 5 | 5 | 3 | 3 |
| Mean | | — | 2.48 | 19.0 | 0.108 | 6.98 |
| SD | | — | 1.27 | 14.7 | 0.0370 | 2.46 |
| Min | | 0.5 | 1.17 | 4.18 | 0.0721 | 4.75 |
| Median | | 2 | 2.51 | 18.1 | 0.105 | 6.58 |
| Max | | 2 | 4.11 | 43.1 | 0.146 | 9.62 |
| CV(%) | | — | 51.2 | 77.2 | 34.3 | 35.2 |
| 108 | Micap3 | 0.5 | 3.46 | 5.01 | 0.233 | 2.97 |
| 109 | Micap3 | 0.5 | 1.28 | 4.24 | 0.184 | 3.77 |
| 110 | Micap3 | 0.5 | 1.45 | 12.9 | 0.0979 | 7.08 |
| 111 | Micap3 | 1 | 1.11 | 8.69 | 0.0606 | 11.4 |
| 112 | Micap3 | 1 | 3.39 | 15.8 | 0.0845 | 8.21 |
| N | | 5 | 5 | 5 | 5 | 5 |
| Mean | | — | 2.14 | 9.33 | 0.132 | 6.69 |
| SD | | — | 1.18 | 4.99 | 0.0732 | 3.43 |
| Min | | 0.5 | 1.11 | 4.24 | 0.0606 | 2.97 |
| Median | | 0.5 | 1.45 | 8.69 | 0.098 | 7.08 |
| Max | | 1 | 3.46 | 15.8 | 0.233 | 11.4 |
| CV(%) | | — | 55.2 | 53.5 | 55.5 | 51.4 |

Median is calculated for Tricor Tmax.

We claim:

1. An encapsulated product comprising
a plurality of micro-capsules formed from a plurality of micro-organisms and having a lipophilic active encapsulated and passively retained within said micro-capsules, said lipophilic active not being a natural constituent of said micro-organisms, each of said micro-capsules having:
(a) an at least substantially intact cell wall; and
(b) an intact cell membrane;
wherein said micro-capsules are formulated to target delivery of said micro-capsules and said lipophilic active to at least one desired mucous membrane.

2. An encapsulated product according to claim 1, wherein said micro-capsules are formulated as one of the group consisting of: syrup, sachet, chewable, chewing gum, orodispersible, dispersible effervescent, dispersible tablet, compressed buccal tablet, compressed sublingual tablet, chewable tablet, melt-in-the-mouth, lozenge, paste, suspension, powder, gel, tablet, compressed sweet, boiled sweet, cream, suppository, snuff spray, aerosol, pessary, and ointment.

3. An encapsulated product according to claim 1, wherein said micro-capsules are formulated within one of a one-part gelatin capsule a two-part gelatin capsule, and an enteric coating.

4. A method of manufacturing an encapsulated product, wherein said encapsulated product comprises a plurality of micro-capsules formed from a plurality of micro-organisms, comprising the step of:
(i) contacting said micro-organisms with a lipophilic active to encapsulate said lipophilic active within said micro-organisms, said lipophilic active being encapsulated and passively retained within said micro-capsules, said lipophilic active not being a natural constituent of said micro-organism, said micro-capsules having:
(a) an at least substantially intact cell wall, and
(b) an intact cell membrane and further comprising the step of:
(ii) formulating said micro-capsules to target delivery of said micro-capsules and said lipophilic active to at least one desired at leas biological membrane.

5. A method of manufacturing an encapsulated product according to claim 4, additionally comprising prior to said encapsulation step, performing at least one treatment step selected from the group consisting of: contacting said micro-organism with an alkaline bleach solution, incubating said micro-organism between 45-60 degrees C., and contacting said micro-organism with a proteolytic enzyme.

6. A method of manufacturing an encapsulated product according to claim 4, additionally comprising after said encapsulation step, performing a conditioning step in which said micro-capsules are incubated in a dry environment between 15-50 degrees C.

7. A method of manufacturing an encapsulated product according to claim 4, wherein said contacting step comprises contacting, for each of said micro-organism, a micro-organism with a lipophilic active in liquid form, said lipophilic active being capable of diffusing into a cell wall of said micro-organism without causing total lysation thereof, the contacting step being carried out in the absence of a lipid extending substance as a solvent or microdispersant for the active and in the absence of a plasmolyser, whereby the active is absorbed by the micro-organism by diffusion across said cell wall and is retained passively within said micro-organism.

8. An encapsulated product according to claim 1, wherein each of said micro-organism is selected from the group consisting of: fungus, bacterium, alga and protozoa.

9. An encapsulated product according to claim 8 wherein said micro-organism is a yeast selected from the taxonomic order Endornycetales.

10. A method of treating a patient comprising administering to said patient a medicament comprising the product of any one of claims 1-3, 8 or 9.

11. A method of manufacturing an encapsulated product according to claim 4, wherein each of said micro-organisms is selected from the group consisting of: fungus, bacterium, alga and protozoa.

12. A method of manufacturing an encapsulated product according to claim 11, wherein said micro-organism is a yeast selected from the taxonomic order Endornycetales.
